(19) United States
(12) Patent Application Publication
(10) Pub. No.: US 2019/0166979 A1
(43) Pub. Date: Jun. 6, 2019

(54) COSMETIC TOOL

(71) Applicant: Sherry Lynn Hale, Grand Island, NY (US)

(72) Inventor: Sherry Lynn Hale, Grand Island, NY (US)

(21) Appl. No.: 15/975,013

(22) Filed: May 9, 2018

Related U.S. Application Data
(60) Provisional application No. 62/594,664, filed on Dec. 5, 2017.

(51) Int. Cl. A45D 44/00; A45D 34/04; A46B 9/02; A46B 15/00; A61B 17/32

(52) U.S. Cl. CPC A45D 44/00; A45D 34/042; A46B 9/021; A61B 2017/320004; A61B 17/32; A45D 2200/25; A46B 2200/1046; A46B 15/0085

(57) ABSTRACT

A cosmetic tool for use in the entire process of the application of permanent makeup. The cosmetic tool provides the following functions: abrading of the epidermis; measuring of the face for proper design placement; precise drawing, dotting, and measuring; stirring of the pigment; and application of temporary pigment to outline the design on the areas of epidermis or skin to be treated prior to application of the permanent makeup; and application of the permanent makeup.

### CROSS-REFERENCE TO RELATED APPLICATIONS

This application is a Nonprovisional of U.S. Provisional Application No. 62/594,664, filed Dec. 5, 2017, the disclosure of which is incorporated herein by reference.

### FIELD OF INVENTION

This invention is directed to a cosmetic tool that can be used in the application of pigment. In particular, this invention is directed to a cosmetic tool for use in the entire process of applying permanent makeup. The cosmetic tool provides the following functions: abrading of the epidermis; measuring of the face for proper design placement; precise drawing, dotting, and measuring; stirring of pigment, and application of pigment to outline the design on the epidermis areas to be treated prior to application of permanent makeup; and application of permanent makeup.

### BACKGROUND OF INVENTION

The permanent makeup industry provides people with tattoos that resemble makeup. Because tattoos permanently pigment the dermis (a layer of the skin that sits below the epidermis), tattoos of eyeliner, eyebrows, eye shadow, lipstick, and other designs will provide a person with permanent makeup.

Before the permanent makeup artist tattoos the dermis, there are several steps that must be taken, all of which use separate or standalone tools. First, he or she abrades the epidermis (the top layer of the skin) with a standalone tool that has a course surface. Abrading the skin is done to remove excess dry skin and to also open the pores of the skin to apply liquid anesthetic that only works on abraded skin. Abrading the skin prior to applying the liquid anesthetic provides for quicker numbing than the typical 20 minutes required if a crème anesthetic is used on the surface of the skin.

After the permanent makeup artist determines the appropriate color for the design, he or she will stir the temporary pigment solution with a standalone tool.

The next step is to dot or place points of temporary pigment on the skin to begin the design process. The location of the dots are typically anchor points that are main points of the design such as the point of the brow tail, the cupids bow of the lip, or the peak of the brow. The permanent makeup artist uses a standalone tool to find the proper angles to place the points.

From there, the permanent makeup artist draws a design onto the abraded epidermis using temporary pigment. Currently, the industry standard indicates the use of a grease pencils, pens, and markers to temporarily draw the design onto the epidermis. After the design has been temporarily drawn onto the epidermis, the permanent makeup artist will tattoo the permanent makeup design onto the person.

As can be imagined, the use of grease pencils, pens, and markers can cause surface contamination due to the inability to properly sanitize the grease pencils, pens, and markers. The foreign substance also has substantial risk of causing allergic reactions due to all the dyes and chemicals in these pencils and markers. While there are medical grade single use markers, these are expensive and not typically used in the industry. Additionally, the temporarily drawn design might still be visible after the tattoo has been permanently placed.

Another problem in the permanent makeup industry is that the permanent makeup artist uses several separate tools for each step in the permanent makeup process. For example, there is one tool to abrade the epidermis, a separate tool to measure out the location of the design, and a separate tool to draw or outline the design. As can be imagined, the permanent makeup artist spends a lot of time placing and replacing different tools on the work tray. In many cases, the tools fall on the floor causing further delay and potential contamination issues.

Thus, there is a long felt need in the industry to reduce the number of tools on the work tray and preferably to find a single tool that can handle all of the steps required in the application of permanent makeup.

### SUMMARY OF THE INVENTION

Accordingly, it is the subject of the present invention to provide a cosmetic tool that combines the functions of multiple tools into one tool and also solves the issue of contamination and allergic reaction. The cosmetic tool of the present invention provides the permanent makeup artist with the ability to create more structured and precise marks of the design on the epidermis so as to precisely indicate where the permanent ink is to be placed.

The cosmetic tool of the present invention is subjected to UV in order to sanitize it prior to use and it is single-use. Thus, the cosmetic tool eliminates any contamination issues.

Another benefit of the cosmetic tool of the present disclosure is that it provides for multiple functions, meaning that there are less tools on the work tray.

The measuring function of the cosmetic tool allows for more precision and accuracy. It also prevents skin staining from the initial outline of the design, thereby allowing the person to see the finished permanent makeup design. The abrading component on the cosmetic tool allows quicker topical desensitization to the epidermis areas being treated. The measuring tool and ruler component is a more precise way to map out and determine the actual distance necessary for appropriate permanent ink application.

### BRIEF DESCRIPTION OF THE DRAWINGS

FIG. 1 is a front view of a cosmetic brush.

FIG. 1A is a left side view of the cosmetic brush shown in FIG. 1.

FIG. 1B is a right side view of the cosmetic brush shown in FIG. 1

FIG. 2 is a rear view of the cosmetic brush.

FIG. 3 is a front view of an enlarged portion of the cosmetic brush showing a first handle end.

FIG. 4 is a front view of an enlarged portion of the cosmetic brush showing a second handle end.

### DETAILED DESCRIPTION OF THE INVENTION

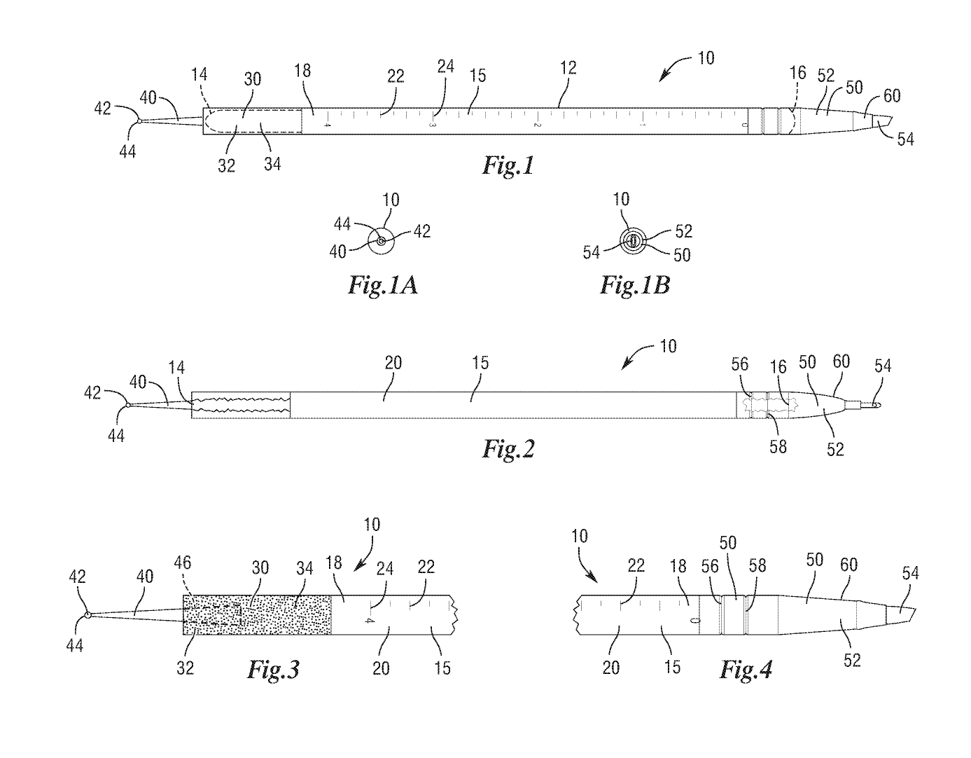

A cosmetic tool 10 is shown in FIGS. 1, 1A, 1B and 2. The cosmetic tool 10 has a handle 12, and the handle 12 has a central handle portion 15 that extends to opposed first and second handle ends 14, 16. In FIG. 1 the first and second handle ends 14, 16 are shown with dashed reference lines, and in FIG. 2 the first and second handle ends 14, 16 are exposed. The central handle portion 15 has an outer handle surface 18 that is cylindrically shaped 20, but in other embodiments the outer handle surface 18 may be differently shaped. The handle 12 may be made of plastic, wood, metal, or stiff plastic, and in one embodiment the handle is solid. The outer handle surface 18 of the central handle portion 15 has printed indicia 22 printed thereon, and the printed indicia 22 includes inch measurement markings 24. As shown in FIG. 1, the inch measurement markings 24 go from zero inches to four inches. In other embodiments the inch measurement markings 24 may be more or less than zero to four inches and may be in centimeters and millimeters in another embodiment.

The cosmetic tool 10 has an abrader 30. The abrader 30 is supported on and is adhered to the first handle end 14 and extends along the central handle portion 15 until it reaches or about reaches the measurement markings 24 that indicates four inches. The measurement markings 24 are useful for allowing a user (not shown) to align the location of pigment placement on the person's skin. The abrader 30 is made of a course material, and this allows a user (not shown) to securely hold the cosmetic brush 10 with his or her fingers (not shown). The abrader 30 also allows the user to scratch or abrade the surface area on which the user is working. For example, the user may scratch the skin of the person being treated. The course material may be applied to the first handle end 14 and part of the central handle portion 15 as a mixture of adhesive 32 and course material 34 that is applied and dried. In another embodiment the abrader 30 may be made of a flexible material and fitted over the first handle end 14 and held in place with a friction fit. In another embodiment the abrader 30 is not present. As shown in FIG. 2, a portion of the abrader 30 has been removed as indicated by the broken line for purposes of illustration to expose a portion of the first handle end 14 and the central handle portion 15.

As best shown in FIG. 3, the cosmetic tool 10 also has a pin 40 that extends from the first handle end 14. The pin 40 has a tip 42 that has a convex surface 44. This allows the pin 40 to be safely used when a user (not shown) is applying cosmetics or pigment (not shown). The pin 40 is used for placing pigment on the person's skin. The pin 40 has an opposed pin end 46 (shown in dashed line in FIG. 3) that is embedded in the central handle portion 15. The pin 40 may be placed in the mold while the central handle portion 15 is being molded. Alternatively, the pin 40 may be heated and pushed through the first handle end 14 and into the central handle portion 15. After cooling the pin 40 is secured to the central handle portion 15.

As best shown in FIG. 4, the cosmetic tool 10 also has a brush assembly 50 that extends from the second handle end 16 and encases a portion of the central handle portion 15. As shown in FIG. 2, a portion of the brush assembly 50 has been removed as indicated by the broken line for purposes of illustration to expose a portion of the second handle end 16 and the central handle portion 15. The brush assembly 50 includes a brush housing 52 and a brush 54 that is supported in the brush housing 52. The brush housing 52 is sized such that it can be fitted over the central handle portion 15 as shown. The brush housing 52 may be made of metal and in one embodiment is press fitted onto the central handle portion 15. After the press fitting is completed first and second circular shaped recessed indentations 56, 58 extend into the central handle portion 15 thus securing the brush assembly 50 to the central handle portion 15. The brush housing 52 extends along central handle portion 15 until it reaches or about reaches the measurement marking 24 that indicates zero inches. As shown, the brush housing 52 has a taper 60 as it extends to the brush 54. This allows a user to see more clearly what he or she is working on. The brush 54 is sloped relative to the brush housing 52 and this allows the user to more accurately draw the permanent makeup design. In one embodiment, the brush 54 has a 45 degree angle and is equal to or less than 1 mm thick. Of course, other brush angles and thicknesses work. The brush housing 52 is made of metal in one of the embodiments, but may also be made of stiff plastic, and in other embodiments the brush housing 52 may be pinned to the central handle portion 15.

It is noted that while brush 54 may have other brush angles and thicknesses that will work, the combination of a brush angle of 45 degrees and brush thickness of equal to or less than 1 mm has been determined to be preferable. After many different combinations were tried, this brush angle and thickness combination was determined to be the best for drawing a design that has a very fine line.

The cosmetic tool 10 of the present invention has the following benefits: it eliminates cross contamination issues because it is single-use and it frees up room on the working tray because it combines six different tools into one. Previously, the working tray had to include a grease pencil or marker, a ruler, an abrading tool, a brush, stirring tools, templates for drawing, brow guide designs, and a measuring caliper or protractor.

The cosmetic tool 10 of the present invention may also be be used by instructors of permanent makeup. The instructor places tip 42 on the top of the skin near the student's needle during tattooing. While the student is inserting the needle and pigment into the skin, the instructor will hold the ball tip 42 very near to the permanent pigment application in order to feel the vibration and pressure of the student's permanent pigment application and then can instruct the student to apply less or more pressure. The instructor can also use tip 42 as a pointing tool for the student during application for precise location to place or apply more permanent pigment. Finally, tip 42 may also be used to unplug a pigment bottle.

Importantly, the cosmetic tool 10 obviates the need for grease pencils, pens, and markers to draw the design onto the skin. Until now, grease pencils, pens, and markers had been the industry standard for marking the person's epidermis with the permanent makeup design. As discussed above, there is no way to properly sanitize a grease pencil, pen, or marker. Because the instruments used to draw the permanent makeup designs weren't properly sanitized, many of the people being treated would develop infections or allergic reactions. It is expected that the cosmetic tool 10 will be a single-use tool and therefore will not cause any cross contamination between people being treated. Additionally, the cosmetic tool 10 is sanitized prior to use by placing it in a UV sterilizer for 15 minutes.

There are many additional disadvantages to the industry standard for marking the permanent makeup design. For example, grease pencils do not allow for the manipulation of line thickness and often the user has to use a knife to maintain the grease pencil's usability. Markers contaminate the skin and are often visible after the services and take time to wear off and thus the person is not able to see the finished permanent makeup immediately. Finally, pencils need sharpeners which are not sterilized and provide areas for continuous bacteria growth.

Further benefits of the cosmetic tool 10 include the ability to create more structured, accurate, and precise marks (drawings of the design) to indicate the areas of application. This is done by both the pin 40 and the brush 54. Because the temporary pigment doesn't stain the skin and wipes off better than the marks left by grease pencils and markers, the person is able to immediately see the finished permanent makeup.

Further features of the cosmetic tool 10 include the elimination of six separate implements on the working tray. Because the cosmetic tool has six functions in one implement, the user can eliminate five of the tools on the work tray, thereby making it much easier to keep track of the cosmetic tool 10 during the service. Moreover, the cosmetic tool saves the permanent makeup artist time by allowing him or her to not have to go back and forth to the tray to change implements during the drawing of the design. Overall, the cosmetic tool 10 allows the user to stir the temporary pigment to be applied to the person's epidermis, abrade the treatment surface or area (typically the person's skin or epidermis), precisely measure the person's face, and draw and dot the marks or outline of the design drawing.

The abrader 30 of cosmetic tool 10 allows the user to abrade the surface area or epidermis or skin, which causes quicker topical desensitization to the areas being treated. Topical anesthetic takes longer to penetrate the skin without abrading, so the abrader provides quicker desensitization. The measurement markings 24 of cosmetic tool 10 offer the user a more precise way to map out and determine the actual distance necessary for appropriate application of the temporary pigment and the permanent makeup. The permanent makeup can be a reconstruction of eye brows, eyeliner, lips, areolas, scalp pigmentation, and scar revision.

The brush 54 provides the ability to draw a fine line by using a certain density and angle of the brush 54. In one embodiment, the brush 54 is made out of synthetic hair. The tip 42 is used to create further definition. The marker measurements 24 allows the user the ability to align distances necessary for symmetry of the permanent makeup. Abrader 30 allows for exfoliation of the surface area to be treated in order for quicker and better absorption of topical anesthetic and temporary pigment. The brush 54 may be used to apply the anesthetic. The pin 40 and tip 42 allows for precise identification of the areas to be treated specific to the symmetry of the person's face. The pin 40 and tip 42 also allows for the stirring of temporary pigment.

It is noted that a user must be trained in the industry of permanent makeup prior to the use of the cosmetic tool 10.

Many individuals in the permanent make up industry are very concerned about cross contamination issues and have expressed the need for a cosmetic tool that provides ease of use and the ability to provide a better result with fewer opportunities for mistakes. Many permanent makeup artists are also concerned with client comfort during the services.

In one embodiment, the cosmetic tool 10 may include a cotton swab area to use to dab the pigment area or wipe the epidermis or skin being treated. In another embodiment, the cosmetic tool 10 includes a pen light.

It is envisaged that artists outside of the permanent makeup industry will find the cosmetic tool 10 to be useful. For example, nail technicians could use the abrader 30 of cosmetic tool 10 to file nails. They could use the brush 54 and tip 42 of cosmetic tool 10 for their artwork application on the nails.

### EXAMPLES

### Example 1

In one embodiment, a method of using a cosmetic tool 10 for permanent eyebrows is provided, wherein the method includes the steps of: abrading the skin of the person with abrader 30; pouring temporary pigment into a reservoir (not shown); stirring the temporary pigment with pin 40 and tip 42; placing temporary pigment onto a glove (not shown); using the measurement markings 24 of cosmetic tool 10 to perfectly align the outline and drawing of the permanent eyebrow makeup; dipping pin 40 and tip 42 into the reservoir of temporary pigment; using pin 40 and tip 42 to outline a permanent eyebrow makeup design on a person's face (not shown); using brush 54 to further draw and define the outline of the permanent eyebrow makeup design; and discarding cosmetic tool 10 when finished.

It will be appreciated by those skilled in the art that while the cosmetic tool has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claims.

What is claimed is:

1. A cosmetic tool comprising:
a handle having a central portion having an outer surface handle and opposed first and second handle ends;
an abrader supported on and adhered to the first handle end extending along the central handle portion;
a pin that extends from the first handle end, wherein the pin has a tip having a convex surface, and wherein the pin is embedded in the central handle portion;
a brush assembly extending from the second handle ends encasing a portion of the central handle portion;
wherein the brush assembly further includes a brush housing sized such that it can be fitted over the central handle portion; and
wherein the brush housing has a taper that extends to a brush.

2. The cosmetic tool of claim 1 wherein the outer handle surface of the central portion of the handle is cylindrical.

3. The cosmetic tool of claim 1 wherein the handle is made from the group consisting of plastic, wood, metal, composite, and combinations thereof.

4. The cosmetic tool of claim 1, wherein the abrader is made of a course material.

5. The cosmetic tool of claim 1, wherein the brush is made of synthetic material.

6. The cosmetic tool of claim 1, wherein the brush has a 45 degree slope relative to the brush housing and has a thickness of 1 mm.

7. The cosmetic tool of claim 1, wherein the brush housing is made of metal and further includes first and second circular shaped recessed indentations that extend into the central handle portion.

8. The cosmetic tool of claim 1, wherein the pin and tip are made of metal.

9. The cosmetic tool of claim 1, wherein the outer handle surface of the central handle portion has printed indicia that includes measurement markings.

10. The cosmetic tool of claim 9, wherein the measurement markings are in inches and are from zero to four inches, wherein the zero inch printed indicia begins on the second handle end and the four inch printed indicia is on the first handle end.

11. The cosmetic tool of claim 2, wherein the outer handle surface of the central portion of the handle is cylindrical.

12. The cosmetic tool of claim 11, wherein the abrader is made of a course material.

13. The cosmetic tool of claim 12, wherein the brush is made of synthetic material.

14. The cosmetic tool of claim 13, wherein the brush has a 45 degree slope relative to the brush housing and has a thickness of 1 mm.

15. The cosmetic tool of claim 14, wherein the brush housing is made of metal and further includes first and second circular shaped recessed indentations that extend into the central handle portion.

16. The cosmetic tool of claim 15, wherein the pin and tip are made of metal.

17. The cosmetic tool of claim 16, wherein the outer handle surface of the central handle portion has printed indicia that includes measurement markings.

18. The cosmetic tool of claim 17, wherein the measurement markings are in inches and are from zero to four inches, wherein the zero inch printed indicia begins on the second handle end and the four inch printed indicia is on the first handle end.

19. A cosmetic tool comprising:
a handle having a central portion having an outer surface handle and opposed first and second handle ends;
a pin that extends from the first handle end, wherein the pin has a tip having a convex surface, and wherein the pin is embedded in the central handle portion;
a brush assembly extending from the second handle ends encasing a portion of the central handle portion;
wherein the brush assembly further includes a brush housing sized such that it can be fitted over the central handle portion; and
wherein the brush housing has a taper that extends to a brush.

20. A cosmetic tool comprising:
a handle having a central portion having an outer surface handle and opposed first and second handle ends;
an abrader supported on and adhered to the first handle end extending along the central handle portion;
a brush assembly extending from the second handle ends encasing a portion of the central handle portion;
wherein the brush assembly further includes a brush housing sized such that it can be fitted over the central handle portion; and
wherein the brush housing has a taper that extends to a brush.
